(19) United States
(12) Patent Application Publication
(10) Pub. No.: US 2021/0128332 A1
(43) Pub. Date: May 6, 2021

(54) LINE PULLER ASSEMBLY FOR AN ENDO-LUMINAL DELIVERY DEVICE

(71) Applicant: Cook Medical Technologies LLC, Bloomington, IN (US)

(72) Inventors: Logan Smith, Mount Gravatt (AU); James Collins, Red Hill (AU); William Parke, Paddington (AU)

(21) Appl. No.: 16/668,135

(22) Filed: Oct. 30, 2019

(51) Int. Cl. A61F 2/95; A61F 2/07

(52) U.S. Cl. CPC A61F 2/95; A61F 2/07; A61F 2002/9522; A61F 2002/9511; A61F 2002/9517

(57) ABSTRACT

A line puller assembly for an endo-luminal delivery device is disclosed. The assembly comprises: a line puller body having a proximal end and a distal end; a line puller actuator assembly mounted to the line puller body for rotation about the line puller body, the actuator assembly including a line deflector ring, the ring including a first deflector line guide; a line anchor location on the line puller body; a first body line guide on the body, the line anchor location and the first body line guide positioned on opposite sides of the ring; and a line having an end portion anchored at the line anchor location on the line puller body, the line extending through the first line deflector ring, whereby a line path extends from the anchor location, through the first deflector line guide and past the first body line guide, the line path having a variable length.

### FIELD OF THE DISCLOSURE

The present disclosure relates to endografts and their delivery systems, sometimes referred to as endo-luminal delivery device assemblies. In particular, the present disclosure relates to endo-luminal delivery device assemblies capable of delivering prostheses, endografts or stent-grafts into the lumen of a vessel.

### BACKGROUND

This disclosure relates generally to medical devices and methods of using the medical devices, and more particularly, to an endo-luminal prosthesis delivery device and methods for placement and deployment of the prosthesis in the lumen of a vessel.

The use of delivery devices or introducers employing catheters has long been known for a variety of medical procedures, including procedures for establishing, re-establishing or maintaining passages, cavities or lumens in vessels, organs or ducts in human and veterinary patients, occlusion of such vessels, delivering medical treatments and other interventions. For these procedures, it has also long been known to deliver an implantable medical device by means of a catheter, often intraluminally. For example, a stent, stent-graft, vena cava filter or occlusion device may be delivered intraluminally from the femoral artery, via a transapical approach and/or using other acceptable delivery locations and methods for deployment of the prosthesis.

For procedures in which a prosthesis or other medical device is implanted into a patient, the prosthesis to be implanted is normally held on a carrier catheter or cannula of the introducer in a compressed state and then released from the cannula so as to expand to its normal operating state, prior to withdrawal of the cannula from the patient to leave the implant in position. In many devices, the steps to carry out the implantation my occur, for example, first by retracting a retractable sheath to expand or partially expand the prosthesis, and then performing further steps to, for example, release one or both ends of the prosthesis, deploy an anchoring stent, or the like. Often diameter reducing ties are employed which requires release using a trigger line, the line normally in the form of a thin wire.

Prosthesis and delivery devices are used in minimally invasive aortic intervention. They are used by surgeons to treat aneurysms and to repair regions of the aorta, including the aortic arch, the thoracic aorta, the abdominal aorta and the aortic bifurcation.

Delivery devices used for minimally invasive aortic intervention allow deployment of intraluminal prostheses or endografts into the lumen of a patient from a remote location.

Numerous devises and procedures have been developed that involve the percutaneous insertion of a prosthesis into a body lumen, such as a blood vessel or duct, of a patient's body. Such a prosthesis may be introduced into the lumen by a variety of known techniques. For example, a wire guide may be introduced into a blood vessel using the Seldinger technique. This technique involves creating a surgical opening in the vessel with a needle and inserting a wire guide into the vessel through a bore of the needle. The needle can be withdrawn, leaving the wire guide in place. A delivery device is then inserted over the wire guide and into the vessel. The delivery device may be used in conventional fashion to insert into the blood vessel a variety of prostheses, such as stents, stent-grafts, catheters, cardiac leads, balloons, and the like.

For example, the delivery device may be used to deliver and deploy an expandable prosthesis, such as a stent-graft, to an aneurysmal blood vessel site. A stent-graft is usually formed from a tubular body of a biocompatible graft material with one or more stents mounted into or onto the tubular body to provide support therefor. The stents may be balloon expandable stents and/or self-expanding stents. The deployment of the prosthesis into the lumen of a patient from a remote location by the use of an introducer delivery and deployment device is described in, for example, U.S. Pat. No. 7,435,253 to Hartley entitled “A Prosthesis and a Method and Means of Deploying a Prosthesis”, which is incorporated herein by reference in its entirety. These, and many other means or devices for delivering endo-luminal grafts typically employ pullable lines in the form of wires. These lines or wires require mechanisms for their actuation.

Endovascular delivery devices require significant expertise and experience to operate. Ease of operation and correct sequencing of various manual operations performed outside the body (at a distal end of a delivery device) are required for successful and optimum deployment of an endograft. It is desirable to make operation as intuitive and foolproof as possible.

Endovascular delivery devices should, where ever possible, avoid catch points and should be robust against misuse or damage.

There is a need to provide an improved endo-luminal delivery device and/or to provide an improved line or wire puller assembly for an endo-luminal delivery device.

While this disclosure may be generally discussed in relation to a delivery device for a stent-graft and method of deployment thereof into one or more specific arteries, including the aorta and iliac arteries, it is also contemplated that the disclosure is not so limited and may relate to any prosthesis and/or any body or vessel lumen in which such a deployment is necessary or desired.

Throughout this document, when referring to a prosthesis delivery device, “proximal” refers to the part of the delivery device that is furthest from the operator and intended for insertion in a patient's body (and usually that part being placed proximal to the target site within the patient) and “distal” refers to that part of the delivery device closest to the operator (and usually that part being placed most distal to the target site within the patient). With regard to the prosthesis being delivered to by the delivery device, the term “proximal” refers to that part of the prosthesis that is closest to the proximal end of the delivery device and “distal” refers to the opposite end of the prosthesis.

### SUMMARY

According to an aspect of the disclosure, a line puller assembly for an endo-luminal delivery device comprises:

a line puller body having a proximal end, a distal end and an intermediate portion between the proximal and distal ends;

a line puller actuator assembly mounted to the line puller body for rotation about the line puller body, the line puller actuator assembly including a line deflector ring located between the proximal and distal ends of the line puller body, the line deflector ring including a first deflector line guide;

a line anchor location on the line puller body;

a first body line guide on the line puller body, the line anchor location and the first body line guide positioned on opposite sides of the line deflector ring; and

a line having an end portion anchored at the line anchor location on the line puller body, the line extending through the first line deflector ring, whereby a line path extends from the anchor location, through the first deflector line guide and past the first body line guide, the line path having a variable length,

wherein rotation of the line puller actuator assembly relative to the line puller body from an initial position to an actuated position lengthens the line path, thereby pulling the line.

In one form, the first deflector line guide of the line deflector ring comprises a first arcuate guide surface.

In one form, the first arcuate guide surface is shaped such that the radius through which the line travels around the first arcuate guide surface does not fall below 3 mm as the line puller actuator assembly is rotated through an initial 45 degrees of rotation relative to the line puller body.

In one form, line puller assembly further comprises a ratchet assembly arranged and constructed to allow rotation of the line deflector ring about the line puller body in a first direction only and to limit rotation about a second direction, the second direction opposite to the first direction.

In one form, the ratchet assembly comprises a pawl depending from the line puller actuator assembly and a gear depending from the line puller body.

In one form, the line puller actuator assembly comprises an actuator frame engaged with the line deflector ring, the actuator frame extending axially over the line deflector ring, whereby an annular cavity is formed between an inner wall of the actuator frame and the line puller body.

In one form, the line puller actuator assembly comprises a ring grip, the ring grip engaged with the actuator frame for rotation therewith and being hand graspable by an operator for actuation of the line puller actuator assembly.

In one form, the ring grip is slidable from: a locked position in which the line puller actuation assembly is locked with respect to the body, to an unlocked position in which the line puller actuation assembly is unlocked with respect to the body.

In one form, the line puller assembly further comprises:

a second deflector line guide in the line deflector ring, the second deflector line guide circumferentially spaced apart from the first deflector line guide; and

a second body line guide on the line puller body,

whereby the line path extends from the anchor location, through the first deflector line guide, past the first body line guide before extending through the second deflector line guide and past the second body line guide.

In one form, the second deflector line guide of the line deflector ring comprises a second arcuate guide surface.

In one form, the second arcuate guide surface is shaped such that the radius through which the line travels around the second arcuate guide surface does not fall below 3 mm as the line puller actuator assembly is rotated through an initial 45 degrees of rotation relative to the line puller body.

In a further aspect of the disclosure, an endo-luminal delivery device comprises:

a handle assembly at a distal end thereof, the handle assembly comprising a line puller assembly as described above;

a tip assembly at a proximal end thereof;

a guide wire catheter extending through the handle assembly, the guide wire catheter being affixed at a distal end thereof to the handle assembly and being affixed at a proximal end thereof to the tip assembly; and

an endograft receiving portion for receiving an endograft distally adjacent to the tip assembly.

In one form, the first deflector line guide of the line deflector ring comprises a first arcuate guide surface.

In one form, the first arcuate guide surface is shaped such that the radius through which the line travels around the first arcuate guide surface does not fall below 3 mm as the line puller actuator assembly is rotated through an initial 45 degrees of rotation relative to the line puller body.

In one form, the line puller assembly further comprises a ratchet assembly, the ratchet assembly arranged and constructed to allow rotation of the line deflector ring about the line puller body in a first direction only and to limit rotation about a second direction, the second direction opposite to the first direction.

In one form, the ratchet assembly comprises a pawl depending from the line puller actuator assembly and a gear depending from the line puller body.

In one form, the line puller actuator assembly comprises an actuator frame engaged with the line deflector ring, the actuator frame extending axially over the line deflector ring, whereby an annular cavity is formed between an inner wall of the actuator frame and the line puller body.

In one form, the line puller actuator assembly comprises a ring grip, the ring grip engaged with the actuator frame for rotation therewith and being hand graspable by an operator for actuation of the line puller actuator assembly.

In one form, the ring grip is slidable from: a locked position in which the line puller actuation assembly is locked with respect to the body, to an unlocked position in which the line puller actuation assembly is unlocked with respect to the body.

In a further aspect of the disclosure, an endo-luminal delivery device comprises:

a handle assembly at a distal end thereof, the handle assembly comprising a line puller assembly comprising:
a line puller body having, a proximal end, a distal end and an intermediate portion between the proximal and distal ends;
a line puller actuator assembly mounted to the line puller body for rotation about the line puller body, the line puller actuator assembly including a line deflector ring located between the proximal and distal ends of the line puller body, the line deflector ring including a first deflector line guide;
a line anchor location on the line puller body;
a first body line guide on the line puller body, the line anchor location and the first body line guide positioned on opposite sides of the line deflector ring;
a line having an end portion anchored at the line anchor location on the line puller body, the line extending through the first line deflector ring, whereby a line path extends from the anchor location, through the first deflector line guide and past the first body line guide, the line path having a variable length;

a tip assembly at a proximal end thereof;

a guide wire catheter extending through the handle assembly, the guide wire catheter being affixed at a distal end thereof to the handle assembly and being affixed at a proximal end thereof to the tip assembly; and

an endograft receiving portion for receiving an endograft distally adjacent to the tip assembly.

### BRIEF DESCRIPTION OF THE DRAWINGS

FIG. 1 shows a delivery device with a handle assembly at a distal end of the device and a prosthesis loaded to the proximal end of the device;

FIG. 2 is a similar view to FIG. 1 but shows a different delivery device having had a plurality of line puller assemblies positioned at its distal end;

FIGS. 3A and 3B are side views of one of the line puller assemblies shown in FIG. 2 in initial and then actuatable positions respectively;

FIG. 3C shows the line puller assembly of FIG. 3A in a perspective view;

FIG. 4A is a side view showing the internal components of the line puller assembly shown in FIG. 3C according to one embodiment of the disclosure;

FIG. 4B is a side view showing the internal components of the line puller assembly shown in FIG. 3C according to a further embodiment of the disclosure;

FIG. 5 is an isometric view of the line puller body of the line puller assembly shown in FIG. 4B;

FIG. 6A is an isometric view showing a line deflector ring which forms part of a line puller actuator assembly of FIGS. 3C, 4B and 5.

FIGS. 6B and 6C are isometric and cross-sectional views respectively of the line deflector ring shown in FIG. 6A;

FIG. 7 is a further isometric view of the line puller body shown in FIG. 5, but also shows a line or wire and its routing;

FIG. 8 is a diagrammatic view showing part of the path of the line shown in FIG. 7 around a distal end of the line puller body shown in FIG. 7;

FIGS. 9A, 9B and 9C are diagrammatic representations showing the progressive changes in the line path shown in FIG. 4A as the line deflector ring is rotated;

FIGS. 10A, 10B and 10C are diagrammatic representations showing the progressive changes in the line path shown in FIG. 4B as the line deflector ring is rotated;

FIG. 11 is a diagrammatic isometric view illustrating the line puller actuator assembly, parts of which can be seen in FIGS. 3A, 3B and 3C and in FIG. 4B;

FIG. 12 is an isometric view showing an actuator frame, the frame being a component of the assembly shown in FIG. 11;

FIG. 13 is a diagrammatic cross-sectional view through the assembly shown in FIG. 11;

FIG. 14 is a similar view to that of FIG. 12 but from a different viewpoint;

FIGS. 15A, 15B, 15C and 15D are diagrammatic isometric views showing operation of the line puller assembly shown in FIGS. 3A, 3B, 3C and 4B; and

FIG. 16 is a graph showing the length of line pulled per unit of rotation of the actuator of the line puller assembly.

### DETAILED DESCRIPTION

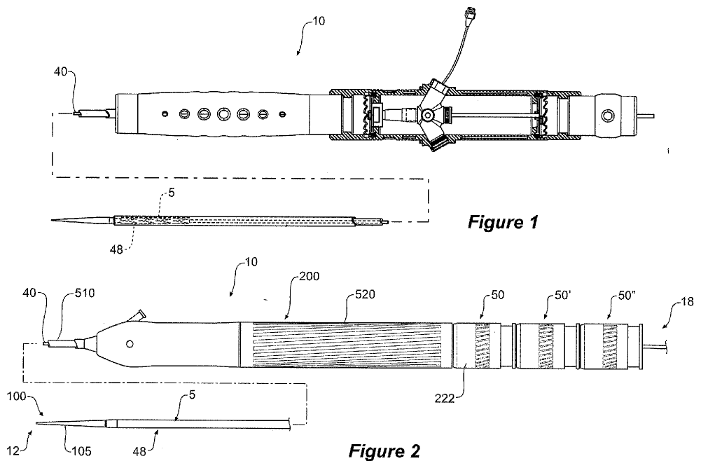

Referring to FIG. 2, an endo-luminal delivery device 10 is shown. The endo-luminal delivery device 10 comprises a handle assembly 200 at a distal end thereof. The handle assembly comprises three line puller assemblies 50. Other endo-luminal delivery devices may have fewer or more line puller assemblies 50. For instance, a single line puller assembly 50 may be adequate in some applications. The endo-luminal delivery device 10 shown in FIG. 2 also comprises a tip assembly 100 at a proximal end 12 thereof. A guide wire catheter 40 extends through the handle assembly 200, the guide wire catheter 40 being affixed at a distal end thereof to the handle assembly 200 and being fixed at a proximal end thereof to the tip assembly 100. This general construction is the same for the alternative endo-luminal delivery device 10 illustrated in FIG. 1. In particular, an endograft receiving portion 48 for receiving an endograft 5 is illustrated in both FIGS. 1 and 2, but the endograft 5 is shown in dotted outline in FIG. 1. The endograft receiving portion 48 is distally adjacent to the tip assembly 100 as is shown in both FIGS. 1 and 2.

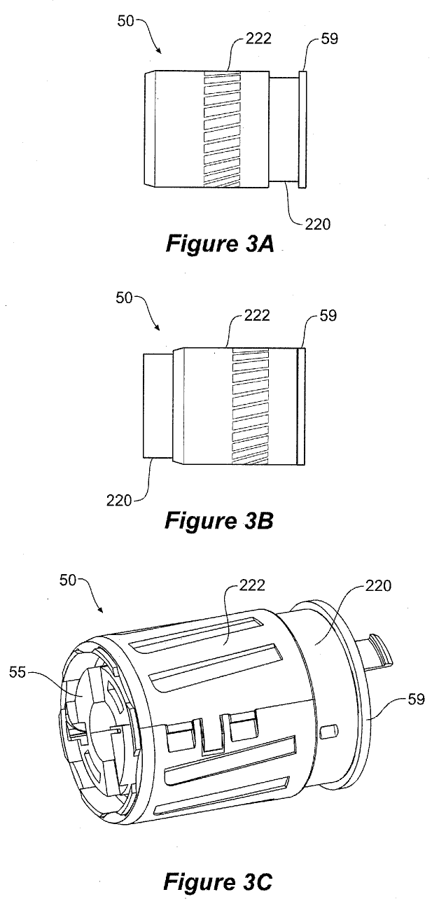

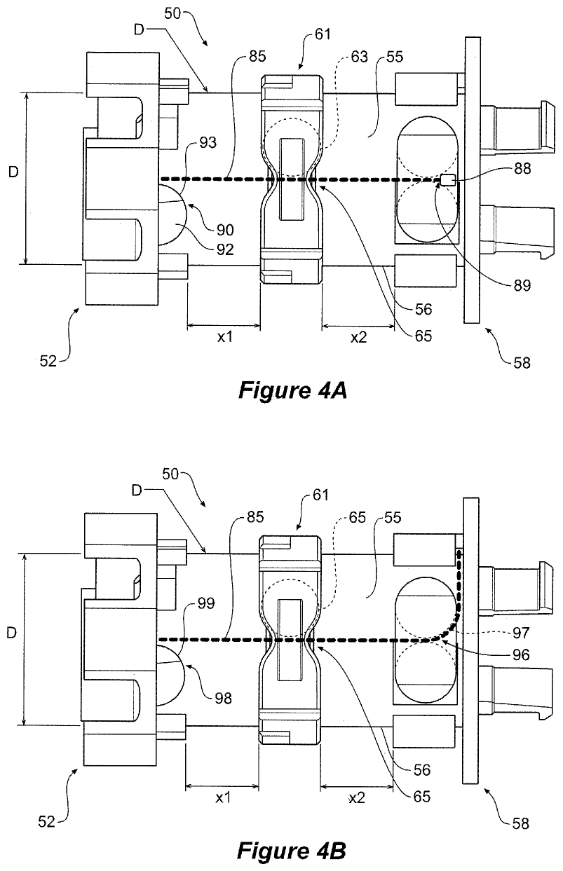

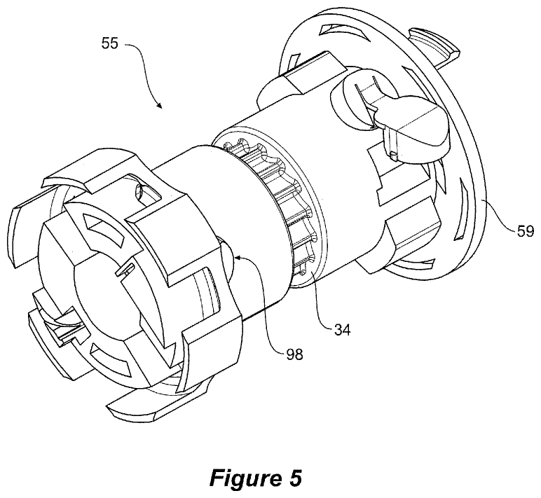

Referring now to FIG. 3A, 3B and 3C, the line puller assembly 50 can be seen in more detail. Referring now to FIG. 3C and FIG. 4A, it can be seen that the line puller assembly 50 comprises a line puller body 55 having a proximal end 52, a distal end 58 and an intermediate portion 56 between the proximal and distal ends 52, 58. The line puller assembly 50 also includes a line puller actuator assembly 60 mounted to the line puller body 55 for rotation about the line puller body 55. The line puller actuator assembly 60 includes a line deflector ring 61 located between the proximal and distal ends 52, 58 of the line puller body 55 as can clearly be seen in FIG. 4A. The line deflector ring 61 includes a first deflector line guide 63, also clearly shown in FIG. 4A.

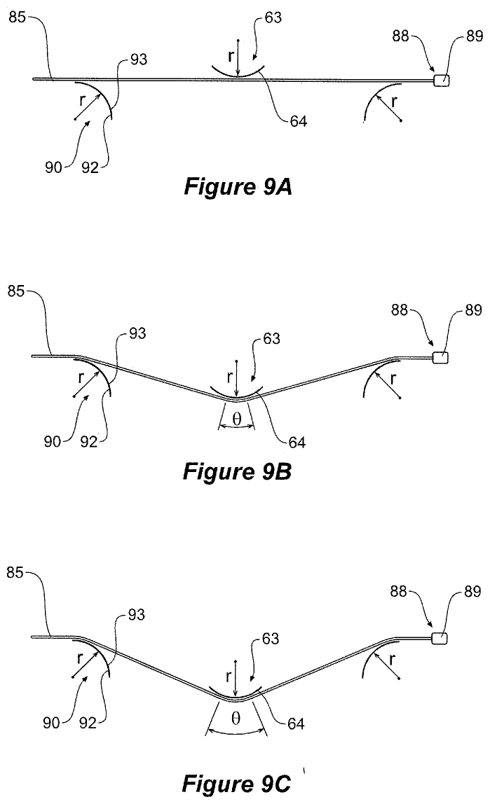

A line anchor location 89 on the line puller body is illustrated in FIG. 4A. Also shown is a first line guide 90 on the line puller body 55. The line anchor location 89 and the first line guide 90 are positioned on opposite sides of the line deflector ring 61. A line 85 having an end portion 88 anchored at the line anchor location 89 on the line puller body 55 can be seen in FIG. 4A. The line 85 extending through the line deflector ring 61 creates a line path. The line path extends from the anchor location 89 through the first deflector line guide 63 and past the first line guide 90 and the line path has a variable length as can be seen in FIGS. 9A, 9B and 9C.

Referring now to FIG. 4A together with FIGS. 9A, 9B and 9C, it can be seen that rotation of the line puller actuator assembly and its line deflector ring 61 lengthens the line path thereby pulling the line 85. FIG. 9A shows an initial line path and then FIG. 9B shows a slightly lengthened line path after the first deflector line guide 63 of the line deflector ring 61 has deflected the line 85 thereby slightly lengthening the line path. Further in FIG. 9C the line path has been lengthened further as the first deflector line guide 63 pushes further into the loop of line 85.

It can be seen that the first deflector line guide 63 of the line deflector ring 61 comprises a first arcuate guide surface 64. This first arcuate guide surface 64 is shown in FIGS. 9A and 9C.

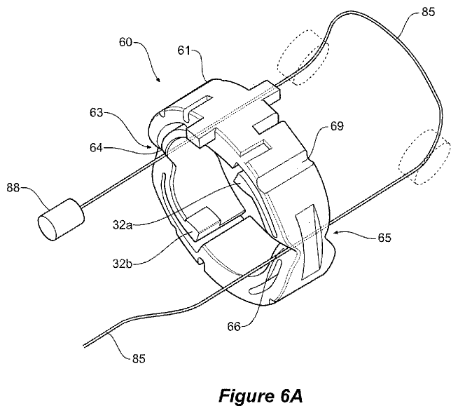

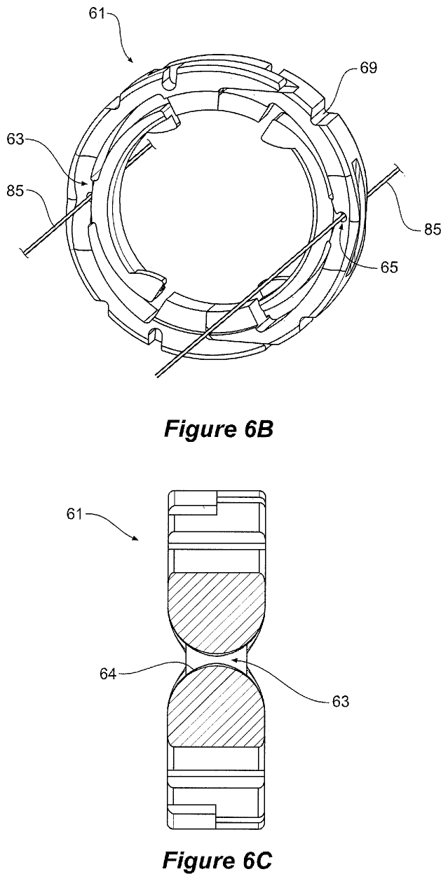

Again referring to FIGS. 9A to 9C, it can be seen that the first arcuate guide surface 64 is shaped such that the radius through which the line travels around the first arcuate guide surface 64 does not fall below 3 mm as the line puller actuator assembly 60 is rotated through an initial 45 degrees of rotation relative to the line puller body. Referring now to FIG. 6C, this cross-sectional view clearly shows the first arcuate guide surface 64. In this embodiment, the first arcuate guide surface 64 is radius with a radius of about 4 mm. Various shapes and radii may be used, including shapes that employ varying radii. It is desirable, however, that at least for a portion of the rotation, that the shape of the first arcuate guide surface 64 is such that the radius through which the line travels around it does not fall below 3 mm. This has the effect of keeping forces and torques within a reasonably range. In some embodiments of the disclosure, radiuses of less than 3 mm may be acceptable. However, there is a trade-off in that as the radius reduces, the friction within the system increases.

So far, the embodiment described is relatively simple in that a line 85 is only deflected by one deflector line guide, the first deflector line guide 63. In a further embodiment of the disclosure, that will now be described in detail, a second deflector line guide 65 is employed. With this embodiment, illustrated in FIGS. 4B, 6A and 6B, it can be seen that the second deflector line guide is circumferentially spaced apart from the first deflector line guide 63. More specifically, in the embodiment illustrated in FIG. 6A, the second deflector line guide 65 is spaced 180 degrees circumferentially apart from the first deflector line guide 63. In other embodiments, the circumferential spacing may be varied. Further, while the embodiments discussed and illustrated have either one or two deflector line guides, in other embodiments, a larger number of deflector line guides may be employed. For instance six or eight line guides may be employed to provide effectively greater gearing within the mechanism.

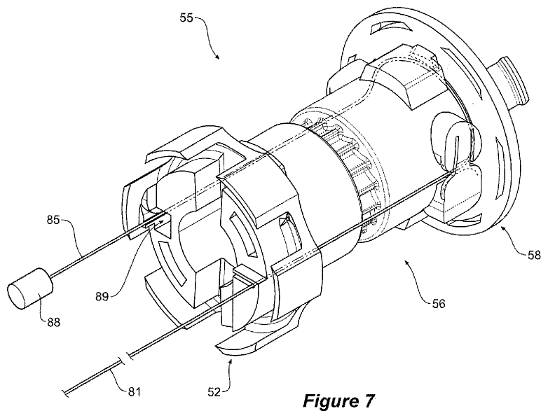

With the line puller assembly illustrated in FIGS. 4B, 6A and 6B, the line path extends from the anchor location 89 illustrated in FIG. 7, through the first deflector line guide 63, past the first body line guide 90 before extending through the second deflector line guide 65 and past the second body line guide 98. The second deflector line guide 65 comprises a second arcuate guide surface 66 as is shown in FIG. 6A.

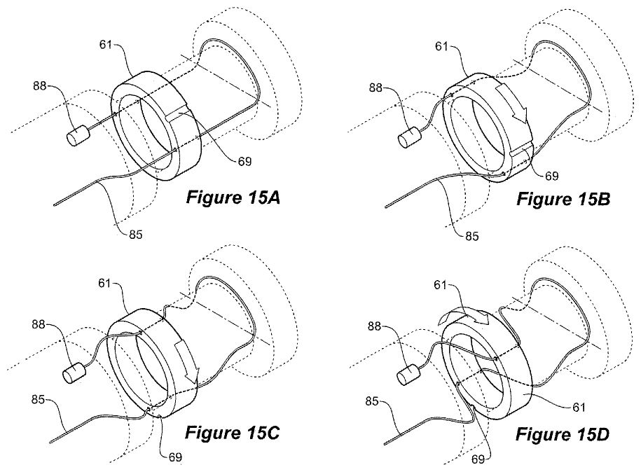

The line puller assembly further comprises a ratchet assembly which is shown in FIG. 5 when read together with FIGS. 6A and 6B. The ratchet assembly is arranged and constructed to allow rotation of the line deflector ring 61 shown in FIG. 6A about the line puller body 55, shown most clearly in FIG. 5, in a first direction, indicated by the arrows in FIGS. 15B, 15C and 15D only and to limit rotation about a second direction, the second direction opposite to the first direction.

Again referring to FIGS. 5 and 6A, it can be seen that the ratchet assembly comprises a pawl 32 depending from the line puller actuator assembly 60 and a gear or rack 34 depending from the line puller body 55. Referring again to FIG. 6A, it can be seen that two pawls 32a and 32b are employed. In other embodiments, a single pawl or three or more pawls may be used.

In other embodiments of the disclosure, the pawl or pawls can be located on the line puller body and the gear or rack may be located on an element of the actuator assembly.

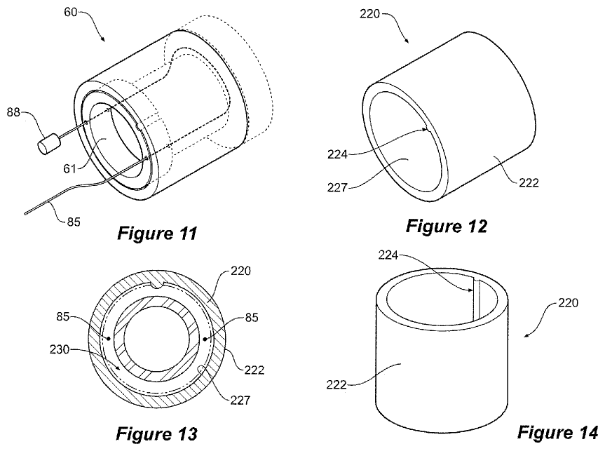

The line puller actuator assembly 60 will now be described in more detail with reference to FIGS. 11, 12, 13 and 14. The line puller actuator assembly 60 includes an actuator frame 220 most clearly shown in a diagrammatic representation of FIGS. 12 and 14. This actuator frame 220 is engaged with the line deflector ring 61 as is illustrated in FIGS. 11 and 13. The actuator frame 220 extends axially over the line deflector ring 61 thereby forming an annular cavity 230. The annular cavity 230, best seen in the cross-sectional view of FIG. 13, is formed between an inner wall 227 of the actuator frame 220 and the line puller body 55. In the embodiment of the disclosure illustrated in FIGS. 4A and 4B, the annular cavity 230 comprises two annular cavities, one each side of the line deflector ring 61.

A key 224, best shown in FIGS. 12 and 14 is provided to engage with a corresponding keyway 69, shown in FIG. 6A so as to operatively couple the actuator frame 220 to the line deflector ring 61 for rotation. Now referring to FIG. 3C, a further element of the line puller actuator assembly 60 is shown. This further element, ring grip 222, is hand graspable by an operator for actuation of the line puller actuator assembly 60. The provision of a separate outer ring grip 222 allows for an additional optional locking feature to be added to the actuator assembly. Referring to FIGS. 3A and 3B, it can be seen that the ring grip 222 is slidable from the position shown in FIG. 3A to the position shown in FIG. 3B. In the position shown in FIG. 3A, the line puller actuator assembly 60 is locked with respect to the body 55. In contrast, in the position shown in FIG. 3B, the line puller actuator assembly 60, and its line deflector ring 61, is rotatably unlocked with respect to the body 55. This allows an operator, such as a surgeon, to actuate the line puller assembly 50, but only after positively sliding the ring grip 222 axially in a distal direction from the position shown in FIG. 3A to the position shown in FIG. 3B.

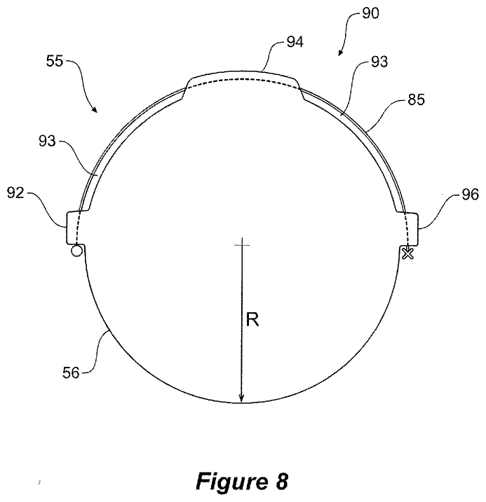

The line path for line 85 will now be described in more detail with reference to FIGS. 2, 4B, 6A, 7 and 8 and 10A, 10B and 10C. Referring first to FIG. 7, the line anchor 88 within the body 55 is shown. The line 85 extends from this position, being held by the end portion 88, through the line deflector ring 61 as is shown in FIG. 6A. From there, the line 85 continues around the first line guide 90 which is shown in FIG. 8. The line 85 is curved around a first line guide protrusion 92 then around the major diameter D of the body 55 (D is shown in FIG. 4B) being guided by a further first line guide protrusion 94 and then is guided back in an at least partially proximal direction by a further first line guide protrusion 96. Together the first body line guide protrusions 92, 94 and 96 form the first body line guide 90. It can be seen that the line path includes roughly 180 degrees around a radius R as shown on FIG. 8, R being 0.5D shown on FIG. 4B. From the position indicated by an X on FIG. 8, the line continues in a proximal direction through the line deflector ring 61 and to the second body line guide 98. The line 85 then continues towards a proximal end 81 illustrated in FIG. 7, the proximal end 81 terminating in or adjacent to the endograft receiving portion 48 shown in FIG. 2.

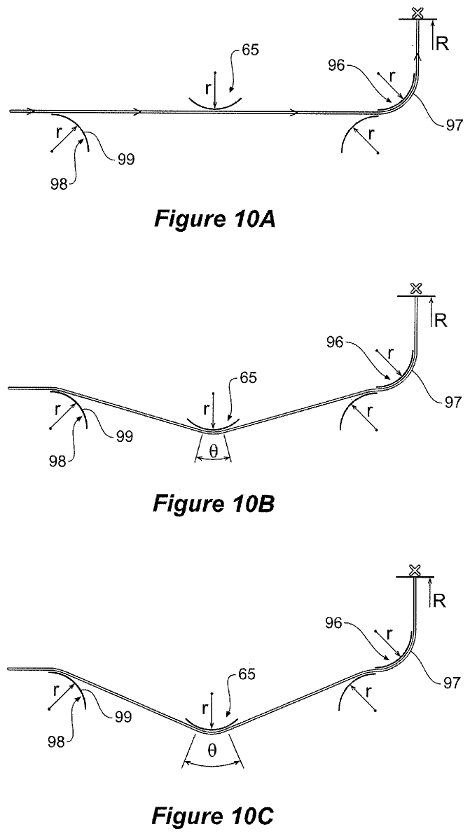

Operation of the line assembly 50 will now be described with reference to FIGS. 10A, 10B and 10C and in relation to the isometric FIGS. 15A, 15B, 15C and 15D. The operator, for instance a vascular surgeon, commences actuation of the line 85 by first sliding the ring grip 222 from the position shown in FIGS. 2 and 3A to the position shown in FIG. 3B. In this position, where the ring grip 222 is hard up against the flange 59, it can be rotated progressively in the direction of the arrows shown in FIGS. 15B, 15C and 15D from an initial position shown in FIG. 15A. As is clear from the progressive FIGS. 15A to 15D, the line path for the line 85 increases in length with rotation of the line deflector ring 61, which is rotated through rotation of the ring grip 222 by the operator. Importantly, the proximal end 81 of the line 85 is moved in a distal direction by a significant distance with significantly less than 360 degrees of rotation of the line puller actuator assembly 60. This means that the operator, for instance a vascular surgeon, need not repeatedly grip rotate, grip rotate and grip rotate in order to achieve sufficient displacement of the wire proximal end 81. With this actuator assembly, there is also a decoupling of the axis of motion which tends to reduce accidental axial movement of the delivery system during high force wire or line retraction. The “gearing” provided by the line actuator assembly 60 also means that it is possible to create a delivery device whereby the ring grip 222 needs to be rotated less than 360 degrees. This has advantages in that it allows indicator elements to be included and also provides the option of having a hard stop to control the degree of rotation (for instance the degree of rotation can be limited to 300 degrees or anything less than 360 degrees). The hard stop can comprise a simple fixed geometry elements, for instance one element may be on on a rotating component and another on a fixed component.

The line guides on the body 55 described above are provided by protrusions. In other embodiments, the line guides on the body 55 may be grooves or other elements for guiding the line 85.

In many applications of this disclosure, the line 85 will be a thin wire 85. In such applications the line puller may be better described as a wire puller. Specifically, in many applications trigger wires for diameter reducing ties, proximal and distal releases may be employed. The term line is used to include non-limiting examples of single strand wire, multi-strand wire, plastic cord or other elongate line of any suitable type.

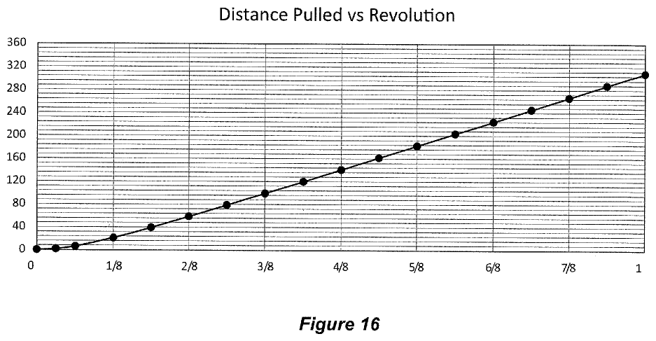

The disclosure addresses a problem with existing delivery devices that employ rotating actuators, such as spools. Specifically, devices that employ a rotating actuator for retracting actuation wires (such as the diameter reducing tie wires, proximal and distal wires and/or tip retrieving wires) sometimes require a large degree of rotation to achieve the required wire pull, in some cases more than 360 degree. For instance, to achieve 250 mm of wire pull with less than 360 degrees of rotation would require unacceptably large diameter spools with current designs. Larger spools would make the handle too bulky and unwieldy. Embodiments of the disclosure described above address the problem of how to pull back a longer length (in the order of 250 mm) while only requiring the operator to rotate the actuator a ‘reasonable’ amount (less than 360 degrees). This can be seen in FIG. 16. FIG. 16 is a graph showing the length of line pulled per unit of rotation of the actuator of the line puller assembly. Just ⅞'s (315 degrees) of one full revolution of the line deflector ring 61 provides 280 mm of wire pull.

Referring again to FIG. 16, it can be seen that the initial rotation on the far left of the graph produces only a very small amount of line displacement. Therefore the operator is applying only a very small amount of initial torque to achieve a high pulling force to overcome the initial static friction within the system. Once ⅛ of a rotation has been achieved (for example), the line pull rate (per degree of rotation) becomes more constant.

The line puller assembly shown in FIGS. 4B and 10A to 10C and in FIGS. 15A to 15D provides for approximately four (4) times the circumference of the line puller body 55 of line (wire) to be wound within one revolution of the line deflector ring 61. The circumference of the body 55 is πD, where D is the diameter of line puller body 55 as is indicated on FIG. 4B.

The ‘gearing’ of approximately four (4) times described above is achieved with just two deflector line guides. In other embodiments, a larger number of deflector line guides may be employed. For instance, six or eight line guides may be employed to provide effectively greater gearing within the mechanism.

An additional benefit of the disclosure is that less than 360 degrees of rotation is required to achieve the required pull, which means that simple hard stops and indicators can be used without the need for complex mechanical switches that would otherwise be required in mechanisms where more than a full revolution (greater than 360 degrees) is needed.

Throughout this specification, unless the context requires otherwise, the words “comprise” and “include” and variations such as “comprising” and “including” will be understood to imply the inclusion of an item or group of items, but not the exclusion of any other item or group items. While various embodiments have been described, it will be apparent to those of ordinary skill in the art that many more examples and implementations are possible within the scope of the present disclosure. Furthermore, although various indications have been given as to the scope of this present disclosure, the present disclosure is not limited to any one of these but may reside in two or more of these combined together. Accordingly, the present disclosure is not to be restricted except in light of the attached claims and their equivalents.

1. A line puller assembly for an endo-luminal delivery device comprising:
a line puller body having a proximal end, a distal end and an intermediate portion between the proximal and distal ends;
a line puller actuator assembly mounted to the line puller body for rotation about the line puller body, the line puller actuator assembly including a line deflector ring located between the proximal and distal ends of the line puller body, the line deflector ring including a first deflector line guide;
a line anchor location on the line puller body;
a first body line guide on the line puller body, the line anchor location and the first body line guide positioned on opposite sides of the line deflector ring; and
a line having an end portion anchored at the line anchor location on the line puller body, the line extending through the first line deflector ring, whereby a line path extends from the anchor location, through the first deflector line guide and past the first body line guide, the line path having a variable length,
wherein rotation of the line puller actuator assembly relative to the line puller body from an initial position to an actuated position lengthens the line path, thereby pulling the line.

2. The line puller assembly of claim 1 wherein the first deflector line guide of the line deflector ring comprises a first arcuate guide surface.

3. The line puller assembly of claim 2 wherein the first arcuate guide surface is shaped such that the radius through which the line travels around the first arcuate guide surface does not fall below 3 mm as the line puller actuator assembly is rotated through an initial 45 degrees of rotation relative to the line puller body.

4. The line puller assembly of claim 1 comprising a ratchet assembly, the ratchet assembly arranged and constructed to allow rotation of the line deflector ring about the line puller body in a first direction only and to limit rotation about a second direction, the second direction opposite to the first direction.

5. The line puller assembly of claim 5 wherein the ratchet assembly comprises a pawl depending from the line puller actuator assembly and a gear depending from the line puller body.

6. The line puller assembly of claim 1 wherein the line puller actuator assembly comprises an actuator frame engaged with the line deflector ring, the actuator frame extending axially over the line deflector ring, whereby an annular cavity is formed between an inner wall of the actuator frame and the line puller body.

7. The line puller assembly of claim 6 wherein the line puller actuator assembly comprises a ring grip, the ring grip engaged with the actuator frame for rotation therewith and being hand graspable by an operator for actuation of the line puller actuator assembly.

8. The line puller assembly of claim 7 wherein the ring grip is slidable from: a locked position in which the line puller actuation assembly is locked with respect to the body, to an unlocked position in which the line puller actuation assembly is unlocked with respect to the body.

9. The line puller assembly of claim 2 comprising:
a second deflector line guide in the line deflector ring, the second deflector line guide circumferentially spaced apart from the first deflector line guide; and
a second body line guide on the line puller body,
whereby the line path extends from the anchor location, through the first deflector line guide, past the first body line guide before extending through the second deflector line guide and past the second body line guide.

10. The line puller assembly of claim 9 wherein the second deflector line guide of the line deflector ring comprises a second arcuate guide surface.

11. The line puller assembly of claim 10 wherein the second arcuate guide surface is shaped such that the radius through which the line travels around the second arcuate guide surface does not fall below 3 mm as the line puller actuator assembly is rotated through an initial 45 degrees of rotation relative to the line puller body.

12. An endo-luminal delivery device comprising:
a handle assembly at a distal end thereof, the handle assembly comprising a line puller assembly as claimed in claim 1;
a tip assembly at a proximal end thereof;
a guide wire catheter extending through the handle assembly, the guide wire catheter being affixed at a distal end thereof to the handle assembly and being affixed at a proximal end thereof to the tip assembly; and
an endograft receiving portion for receiving an endograft distally adjacent to the tip assembly.

13. The line puller assembly of claim 12 wherein the first deflector line guide of the line deflector ring comprises a first arcuate guide surface.

14. The line puller assembly of claim 13 wherein the first arcuate guide surface is shaped such that the radius through which the line travels around the first arcuate guide surface does not fall below 3 mm as the line puller actuator assembly is rotated through an initial 45 degrees of rotation relative to the line puller body.

15. The line puller assembly of claim 12 comprising a ratchet assembly, the ratchet assembly arranged and constructed to allow rotation of the line deflector ring about the line puller body in a first direction only and to limit rotation about a second direction, the second direction opposite to the first direction.

16. The line puller assembly of claim 15 wherein the ratchet assembly comprises a pawl depending from the line puller actuator assembly and a gear depending from the line puller body.

17. The line puller assembly of claim 12 wherein the line puller actuator assembly comprises an actuator frame engaged with the line deflector ring, the actuator frame extending axially over the line deflector ring, whereby an annular cavity is formed between an inner wall of the actuator frame and the line puller body.

18. The line puller assembly of claim 17 wherein the line puller actuator assembly comprises a ring grip, the ring grip engaged with the actuator frame for rotation therewith and being hand graspable by an operator for actuation of the line puller actuator assembly.

19. The line puller assembly of claim 18 wherein the ring grip is slidable from: a locked position in which the line puller actuation assembly is locked with respect to the body, to an unlocked position in which the line puller actuation assembly is unlocked with respect to the body.

20. An endo-luminal delivery device comprising:
a handle assembly at a distal end thereof, the handle assembly comprising a line puller assembly comprising:
a line puller body having, a proximal end, a distal end and an intermediate portion between the proximal and distal ends;
a line puller actuator assembly mounted to the line puller body for rotation about the line puller body, the line puller actuator assembly including a line deflector ring located between the proximal and distal ends of the line puller body, the line deflector ring including a first deflector line guide;
a line anchor location on the line puller body;
a first body line guide on the line puller body, the line anchor location and the first body line guide positioned on opposite sides of the line deflector ring;
a line having an end portion anchored at the line anchor location on the line puller body, the line extending through the first line deflector ring, whereby a line path extends from the anchor location, through the first deflector line guide and past the first body line guide, the line path having a variable length;
a tip assembly at a proximal end thereof;
a guide wire catheter extending through the handle assembly, the guide wire catheter being affixed at a distal end thereof to the handle assembly and being affixed at a proximal end thereof to the tip assembly; and
an endograft receiving portion for receiving an endograft distally adjacent to the tip assembly.
